(19) United States
(12) Patent Application Publication
(10) Pub. No.: US 2026/0092254 A1
(43) Pub. Date: Apr. 2, 2026

(54) FULLY DEFINED MATRICES FOR ORGANOID CULTURE

(71) Applicant: inSoma Bio, Inc., Durham, NC (US)

(72) Inventor: Stefan ROBERTS, Raleigh, NC (US)

(21) Appl. No.: 19/113,814

(22) PCT Filed: Sep. 21, 2023
(86) PCT No.: PCT/US2023/074739

§ 371 Date: Mar. 20, 2025

Related U.S. Application Data
(60) Provisional application No. 63/408,715, filed on Sep. 21, 2022.

(51) Int. Cl. C12N 5/00; A61L 27/38; C12N 5/077

(52) U.S. Cl. CPC C12N 5/0068; A61L 27/3834; C12N 5/0012; C12N 5/0062; C12N 5/0652; A61L 2400/06; C12N 2533/50; C12N 2535/00

(57) ABSTRACT

The present disclosure relates to a composition for three-dimensional cell and/or organoid culture, comprising a phase-changing matrix comprising a partially ordered polypeptide, and a plurality of cells encapsulated in the matrix. In embodiments, the plurality of cells comprises one or more cell types, including at least one cell type selected from the group consisting of: bone-marrow derived mesenchymal stem cells and adipose-derived mesenchymal stem cells. In embodiments, the one or more cell types comprises mesenchymal stem cells, the partially ordered polypeptide comprises at least one chemically-interactive sequence such as arginylglycylaspartic acid (RGD) sequence, the matrix forms a porous network when heated above a transition temperature, and the partially-ordered polypeptide comprises a plurality of structured domains, and a plurality of disordered domains.

### CROSS-REFERENCE TO RELATED APPLICATIONS

This application claims the benefit of U.S. Provisional Patent Application No. 63/408,715, filed on Sep. 21, 2022, the content of which is herein incorporated by reference in its entirety.

### STATEMENT REGARDING FEDERALLY SPONSORED RESEARCH

This invention was made with government support under R43DK129121 awarded by National Institute of Diabetes and Digestive and Kidney Diseases. The government has certain rights in the invention.

### FIELD OF THE DISCLOSURE

The present disclosure relates to three-dimensional cell culture.

### BACKGROUND

Three-dimensional cell culture has become a powerful means of replicating in vivo physiology under controlled in vitro conditions. Substrates for three-dimensional culture, such as Matrigel, remain undefined and highly variable, however, leading to a wide variation in resulting cellular phenotypes. A new option for more defined cell culture conditions is urgently required.

### BRIEF SUMMARY

According to an embodiment, the present disclosure relates to a composition for three-dimensional cell and/or organoid culture, comprising a phase-changing matrix comprising a partially ordered polypeptide, and a plurality of cells encapsulated in the matrix. In embodiments, the plurality of cells comprises one or more cell types, the one or more cell types comprises at least one cell type selected from the group consisting of: bone-marrow derived mesenchymal stem cells and adipose-derived mesenchymal stem cells, the one or more cell types comprises mesenchymal stem cells, the partially ordered polypeptide comprises at least one chemically-interactive sequence such as arginylglycylaspartic acid (RGD) sequence, the at least one chemically-interactive sequence being incorporated from the N terminus and/or the C terminus of the partially ordered polypeptide, the at least one chemically-interactive sequence being incorporated between the N terminus and the C terminus of the partially ordered polypeptide, the matrix forms a porous network when heated above a transition temperature, a concentration of the partially ordered polypeptide within the porous network is between about 0.2 mg/ml and about 300 mg/ml, a void volume of the porous network is between about 60% and about 90%, a void volume of the porous network is substantially unchanged with the helical content of the partially ordered polypeptide is varied between about 12.5% and about 50%, the partially-ordered polypeptide comprises a plurality of structured domains, and a plurality of disordered domains, the partially ordered polypeptide is modified to encode a photoreactive unnatural amino acid into at least one of the plurality of unstructured domains, at least one of the plurality of disordered domains comprises a plurality of an amino acid sequence of (GXGVP)ₙ, X is any amino acid except proline and n is an integer greater than or equal to 1, and the structured domain comprises a polyalanine domain, the disordered domain comprises a plurality of an amino acid sequence of (GXGVP)ₙ, X is Val, or Ala, or a mixture of Ala and Val, and wherein n is an integer from 1 to 50, X is an alternating iteration of Ala and Val in a ratio from 10:1 to 1:10 (Ala:Val), X is an alternating iteration of Ala and Val in a ratio of 1:1 or 1:4, the polyalanine domain comprises (Ala)ₘ, wherein m is an integer from 5 to 50, the polyalanine domain comprises one or more of (A)₂₅, K(A)₂₅K, D(A)₂₅K, GD(A₂₅)K, or GK(A₂₅)K, the partially-ordered polypeptide comprises [(GXGVP)ₙ-GX¹(A)₂₅X¹]ₘ, where X is A or V; X¹ is K or D, n is an integer from 10 to 20, and m is an integer from 4 to 8, the partially-ordered polypeptide comprises one or more of M[(GVGVP)₁₅-GD(A₂₅)K]₆-GWP, M[(GVGVP)₁₅-GD(A₂₅)K]₄-GWP, M[(GVGVP)₁₅-GK(A₂₅)K]₆-GWP, M[(GVGVP)₁₅-GK(A₂₅)K]₄-GWP, M[(G[A₁:V₁]GVP)₁₆-GD(A₂₅)K]₆-GWP, M[(G[A₁:V₁]GVP)₁₆-GD(A₂₅)K]₄-GWP, M[(G[V₄:A₁]GVP)₁₅-GD(A₂₅)K]₆-GWP, or M[(G[V₄:A₁]GVP)₁₅-GD(A₂₅)K]₄-GWP, the partially-ordered polypeptide comprises one or more of M[(GVGVP)₁₅-GD(A₂₅)K]₆-GWP, or M[(G[V₄:A₁]GVP)₁₅-GD(A₂₅)K]₆-GWP, the matrix has a transition temperature of heating (Tt-heating) and a transition temperature of cooling (Tt-cooling), the transition temperature of cooling (Tt-cooling) is concentration-independent, the transition temperature of heating (Tt-heating) and the transition temperature of cooling (Tt-cooling) range from about 10° C. to about 45° C., the matrix forms a solid aggregate above the Tt-heating, the solid aggregate resolubilizes when cooled to below the Tt-cooling, the solid aggregate is a stable three-dimensional matrix, the solid aggregate comprises a plurality of micropores, the plurality of micropores range in size from about 1 pm to about 150 pm, the composition comprises between about 200 pM and about 2 mM of the matrix, the composition comprises the matrix and the plurality of cells in a ratio ranging from about 1:9 to about 9:1, the composition comprises the matrix and the plurality of cells in a ratio ranging from about 1:3 to about 3:1, the composition comprises the matrix and the plurality of cells in a ratio of about 1:1, the composition is a shapeable liquid or semisolid, the composition is injectable or implantable, the composition is shapeable or moldable into 2- or 3-dimensional shapes, areas, or volumes, the matrix permits cell infiltration and vascularization, and/or the cultured plurality of cells forms an organoid.

According to an embodiment, the present disclosure further relates to a method of three-dimensional cell culture, comprising mixing, at a first temperature, a plurality of cells with a phase-changing matrix comprising a partially ordered polypeptide, heating the mixture to a second temperature that is higher than the first temperature, wherein the phase-changing matrix forms a porous network when heated to the second temperature, the porous network having a three-dimensional structure comprising the phase-changing matrix and the plurality of cells, the plurality of cells being encapsulated therein, and culturing the solid aggregate at physiological conditions.

According to an embodiment, the present disclosure further relates to a method of extracting a plurality of cells from three-dimensional cell culture, comprising cooling, to a first temperature, a mixture of the plurality of cells and a phase-changing matrix comprising a partially ordered polypeptide, the phase-changing matrix solubilizing at the first temperature, and extracting the plurality of cells from the solubilized phase-changing matrix.

According to an embodiment, the present disclosure further relates to a method for tissue replacement and/or augmentation, comprising mixing, at a first temperature, a plurality of cells with a phase-changing matrix comprising a partially ordered polypeptide, heating the mixture to a second temperature that is higher than the first temperature, wherein the phase-changing matrix forms a porous network when heated to the second temperature, the porous network having a three-dimensional structure comprising the phase-changing matrix and the plurality of cells, the plurality of cells being encapsulated therein, and implanting the solid aggregate into a subject.

According to an embodiment, the present disclosure further relates to a method for tissue replacement and/or augmentation, comprising mixing, at a first temperature, a plurality of cells with a phase-changing matrix comprising a partially ordered polypeptide, heating the mixture to a second temperature that is higher than the first temperature, wherein the phase-changing matrix forms a porous network when heated to the second temperature, the porous network having a three-dimensional structure comprising the phase-changing matrix and the plurality of cells, the plurality of cells being encapsulated therein, culturing the solid aggregate at physiological conditions, and implanting the solid aggregate into a subject at a target region.

According to an embodiment, the present disclosure further relates to a method for tissue replacement and/or augmentation, comprising mixing, at a first temperature, a plurality of cells with a phase-changing matrix comprising a partially ordered polypeptide, and injecting the mixture into a subject at a target region, the phase-changing matrix forming a porous network when heated at least to a second temperature greater than the first temperature, the second temperature being less than or equal to body temperature, wherein the porous network has a three-dimensional structure comprising the phase-changing matrix and the plurality of cells, the plurality of cells being encapsulated therein.

According to an embodiment, the present disclosure further relates to a method of two-dimensional cell culture, comprising depositing, at a first temperature, a phase-changing matrix comprising a partially ordered polypeptide onto a culture surface, heating the deposited phase-changing matrix to a second temperature, wherein the phase-changing matrix forms a porous network when heated to the second temperature, seeding a plurality of cells onto a surface of the porous network, and culturing the plurality of cells. In embodiments, the method further comprises cooling the phase-changing matrix to the first temperature such that the phase-changing matrix solubilizes and extracting the cultured plurality of cells from the solubilized phase-changing matrix.

### BRIEF DESCRIPTION OF THE SEVERAL VIEWS OF THE DRAWINGS

FIG. 1A shows the incorporation of ordered domains into disordered elastin-like polypeptides, which leads to the formation of microporous protein networks, shown in FIG. 1B, which are reminiscent of native elastin.

FIG. 2A demonstrates phase separation of a POP solution into a porous, solid network at a critical threshold temperature. FIG. 2B demonstrates three-dimensional reconstructed confocal images of networks, revealing that pore size can be tuned with concentration to between 10 μm (at 100 μM concentration) and 500 nm (at 800 μM concentration). FIG. 2C shows that network stiffness can be tuned across more than 4 orders of magnitude by controlling polymer properties.

FIG. 3 illustrates optical density (OD) measurements of a POP as a function of temperature. The graph shows a sharp, reversible phase behavior and hysteresis (ΔTₜ). The Tt-heat and Tt-cool can be independently controlled.

FIG. 4 illustrates the effect of RGD sequences on cell adhesion and proliferation. Human bone marrow-derived mesenchymal stem cells (hMSCs) were cultured within POP matrices with (FIG. 4A, FIG. 4C) and without (FIG. 4B, FIG. 4D) RGD domains. Incorporation of RGD domains enhanced cell spreading within the matrix. FIG. 4E shows that, in addition to cell spreading, inclusion of the RGD domain improved proliferation 5×.

FIG. 5 is a summary of POP compositions developed. Recombinant POPs are comprised of disordered elastin-like polypeptide (ELP) regions and ordered polyalanine helices. 1. POP-80-12.5%, 2. POP-80-25% (referred to as POP-80 for all cell and microcarrier studies), 3. UV-POP-80, 4. UV-RGD-POP-80, 5. RGD-POP-80A, 6. RGD-POP-80B, 7. RGD-POP-80C, 8. RGD-POP-80D, 9. RGD-POP-80E, 10. POP-120, 11. RGD-POP-120.

FIG. 6A shows POPs being successfully encapsulated into microenvironments using water-in-oil microfluidics. When heated, as shown in FIG. 6B and FIG. 6C, POPs form stable, microporous particles that reversibly disassemble upon subsequent cooling. FIG. 6D shows that these particles are monodispersed with pore sizes on the order of 5-10 μm.

FIG. 7A and FIG. 7B demonstrate that POP void volume, and therefore porosity, can be tuned by changing polymer concentration within an apparent range of between 60-90%.

FIG. 7C demonstrates that POP microcarriers created in water in oil microfluidics can be extracted into aqueous solution using a simple washing procedure. FIG. 7D shows that extracted microcarriers are stable and non-interactive, though they are more compact than their unextracted counterparts (SEM scale bar=5 μm).

FIG. 8A demonstrates particle buoyancy. 50 μm POP microparticles settle in physiological solution over several hours. FIG. 8B is a micrograph of a three-dimensional cross section through the particles, revealing a complex internal porous architecture. FIG. 8C shows that POP microcarriers can be made as large as 250.

FIG. 9A through FIG. 9F. Cells may be encapsulated within the POP matrix at Day 0 (FIG. 9A) and remain adhered on the POP network after 8 days in culture (FIG. 9B). Adding an RGD peptide sequence to the C-terminus of POP-80 promotes cell adhesion at Day 0 (FIG. 9D), and greater cell spreading and elongation after 8 days of culture (FIG. 9E). A comparison of 3D renderings of confocal images suggests that unmodified POP (POP-80) remains as a loose network (FIG. 9C), while the RGD fusion (RGD-POP-80A) creates a tightly connected cell sheet (FIG. 9F).

FIG. 10A through FIG. 10F. Cells encapsulated within POP networks at Day 0 on POP-80, 1:1 POP-80:RGD-POP-80A, and RGD-POP-80A (FIG. 10A, FIG. 10B, and FIG. 10C, respectively), vs. at Day 7 on the same (FIG. 10D through FIG. 10F). In order to maintain cell retention within the POP networks, all POP chains comprise at least one RGD sequence.

FIG. 11A through FIG. 11D. Adhesion of hMSCs to the surface of crosslinked UV-RGD-POP-80 networks over 24 hours at 250 μM and 750 μM concentrations (FIG. 11A, FIG. 11B). Comparison between hMSC adhesion over 7 days on crosslinked UV-RGD-POP-80 vs. RGD-POP-80A chemically crosslinked with THPC (FIG. 11C, FIG. 11D). F-actin, depicted with phalloidin, demonstrates cell adhesion and spreading. Scale bars represent 200 μm.

FIG. 12 demonstrates long-term dynamic culture of hMSCs with POP microcarriers. An optimal ratio for long-term culture of hMSCs on POP microcarriers was evaluated by culturing microcarrier suspensions with hMSCs in hMSC growth media on a rocker at 37° C. for 5 days.

FIG. 13A illustrates that the addition of 2 or more external RGD groups promotes cellular adhesion and migration through the POP networks. FIG. 13B shows that cell roundness, wherein a y-axis value of 0 denotes a straight line and a y-axis value of 1 denotes a perfect circle, decreases with the addition of RGD groups onto the ends of the polymer chain compared to unmodified POPs. Shown in FIG. 13C, optical density measurements of POPs at a concentration of 100 μM in PBS demonstrated that addition of external RGD groups increased the heating transition temperature of POPs.

FIG. 14A through FIG. 14F. POPs were designed with RGD motifs within the disordered regions of the polypeptide chains to assess whether location would impact the availability of RGD groups within cell culture. Bone marrow-derived hMSCs were able to adhere to the POP networks, however, reduced adhesion compared to the unmodified POPs and POPs with external RGD groups was detected. POP-80 and RGD-POP-80E are in FIG. 14A and FIG. 14B, and POP-120 and RGD-POP-120 are in FIG. 14D and FIG. 14E, respectively. Optical density measurements performed at a concentration of 100 μM in PBS demonstrated that the heating transition temperature increased substantially with the addition of internal RGD groups for both 80mer and 120mer POPs (FIG. 14C and FIG. 14F).

FIG. 15 is a graphical illustration of cell viability of adipose-derived hMSCs on a variety of POP compositions, as assessed by a commercially available plate-reader assay. These data suggest that two external RGD groups are sufficient to maintain viable cells capable of proliferation within the POP network.

FIG. 16A-C provides multiple illustrations of the effect of UV exposure time on hMSC viability. hMSCs remain viable with exposure times of up to 10 seconds, which is sufficient for full cross-linking of UV-RGD-POP-80. Scale bars represent 200 μm.

FIG. 17 is a graphical illustration of detachment and re-plating of viable hMSCs from cross-linked POP networks. After only one cooling cycle, viable hMSCs can be re-plated from POPs with short cross-linking exposure times.

FIG. 18 is a graphical illustration of percent of cells removed. By varying the density of cross-linking sites within the POP networks by creating mixtures of crosslinkable (UV-RGD-POP-80) and non-UV crosslinkable POP (RGD-POP-80A), an average density of 1 crosslink site per polymer chain was identified as allowing for the highest cell removal compared to completely uncrosslinked POP networks.

FIG. 19A and FIG. 19B provide confocal images of colorectal carcinoma (CRC) enteroids grown on Matrigel and on POPs, respectively, where DAPI is blue and EpCAMA is red. In a head to head pilot study, organoids grown on POPs showed viability with cell bodies indicating formation of an epithelium and EpCAM (a cell adhesion molecule associated with cancer cells) staining observed in the luminal compartment. Some organoids exhibited a potential “apical-out” morphology. In addition to the development of stable organoids on POP matrices, the present disclosure demonstrates significant opportunities for both basal-out and apical-out organoids. Access to the apical epithelium on the external surface of organoids allows screening of orally administered drugs, microbiome compatibility studies, and studies of enteric infection. FIG. 19C provides referential images of basal-out and apical-out arrangements and different CRC morphologies.

### DETAILED DESCRIPTION

### Definitions

The term “a” or “an” refers to one or more of that entity, i.e. can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

As used herein the term “sequence identity” refers to the extent to which two optimally aligned polynucleotides or polypeptide sequences are invariant throughout a window of alignment of residues, e.g. nucleotides or amino acids. An “identity fraction” for aligned segments of a test sequence and a reference sequence is the number of identical residues which are shared by the two aligned sequences divided by the total number of residues in the reference sequence segment, i.e. the entire reference sequence or a smaller defined part of the reference sequence. “Percent identity” is the identity fraction times 100. Comparison of sequences to determine percent identity can be accomplished by a number of well-known methods, including for example by using mathematical algorithms, such as, for example, those in the BLAST suite of sequence analysis programs. Unless noted otherwise, the term “sequence identity” in the claims refers to sequence identity as calculated by Clustal Omega® using default parameters.

Compared to standard two-dimensional methods, three-dimensional cell culture presents an opportunity to more reliably replicate and model in vivo physiology under controlled laboratory conditions. Organoid culture in particular has been shown to replicate in vivo tissue development, differentiation, and disease physiology in many cases. Recently, however, it has been shown that current cell culture substrates, such as a Matrigel, collagen, and other recombinant extracellular matrix components, trigger undesirable phenotypes for modeling normal tissue function using cultured organoids. An unmet need therefore remains for a three-dimensional scaffold, capable of supporting organoid viability while preserving native phenotypes prior to experimental perturbation.

Three-dimensional culture, including organoids representing various tissue types is a rapidly growing method for evaluating cellular physiology, drug activity and holds the potential to revolutionize tissue engineering. Optimizing three-dimensional culture scaffolds will not only improve basic and translational research but will likely accelerate various cell therapeutics and tissue engineering approaches.

Three dimensional cell culture requires a porous, biocompatible matrix to allow cells to grow and migrate to better replicate the in vivo environment compared to standard culture on plastic dishes.

Matrigel is currently the dominant scaffold for three-dimensional culture, but it remains fraught with issues around its undefined nature, batch-to-batch variability, and stimulation of aberrant phenotypes in various cell types, including stem cells. The material is derived from the basement membrane of Engelbreth-Holm-Swarm (EHS) mouse sarcomas, which is then rendered into a polymerizable hydrogel. While the extracellular matrix (ECM) components of Matrigel generally comprise laminin (˜60%), collagen IV (˜30%), entactin (˜8%), and the heparin sulfate proteoglycan perlecan (˜2-3%) (1, 2), proteomic analysis of Matrigel has revealed over 14,000 peptides and nearly 2,000 different proteins. The undefined nature of the material renders interpretation of experiments challenging, with multiple reports identifying this as a concern when using Matrigel. For example, one report identified the growth factor IGF-1 as present at nanogram per milliliter concentrations in Matrigel, while another study found it absent with a concomitant 10-fold difference from batch to batch in growth factors FGF2 and PDGF. In addition to bioactive molecules, Matrigel's biomechanical properties have been shown to be highly variable. Studies have reported firmness of 400-420 Pa and 840 Pa with local regions of the material exhibiting firmness of 1-3 kPa. This issue is not relegated to batch-to-batch variation, with a single polymerized slab of Matrigel reported to have local firmness of 650 Pa and nearly 2 kPa depending on the region evaluated. Finally, the material has been shown to contain viral loads which can impact the behavior of cells such as macrophages. Taken together, there is a clear need for a more defined material for three-dimensional culture if organoids are to continue to support new methods of investigation and drug screening. Providing a more reliable, predictable substrate for three-dimensional culture is likely to enhance the power and control over these studies.

Polyethylene glycol (PEG)-based hydrogels have been shown effective for some workflows in published reports. PEG-based materials offer porous, soft solids with a largely defined structure, except where heparin (a porcine mucosa derived carbohydrate chain of varying sizes) is added. Degradation of PEG hydrogels, however, is challenging for cell/organoid passaging, as a hydrolysis or proteolytic cleavage site must be introduced to allow degradation. Often however, phase transition occurs over time, or with insufficient user control to facilitate workflow steps such as passaging.

Other ECM components, such as laminin and fibronectin, are often used as a tissue culture plate coating for 2D culture. To render them compatible with 3D spheroids, however, they must be mixed with a hydrogel (such as Matrigel itself) or conjugated to hydrogel-forming PEG monomers, the challenges with which are described above. Collagen is also a popular substrate for 3D culture. However, because cell culture collagen is derived from rat tail tendon, it is also not a defined product, similar to Matrigel.

An ideal solution would be easily synthesized at large scale, “defined” in composition without major batch-to-batch variation of biological factors and allow phase-transition from liquid to porous solid for easy scaffold cell seeding.

As described herein, the present disclosure provides a fully defined recombinant protein polymer which meets these criteria. Partially ordered polymers, or partially ordered polypeptides (POPs), are a fully defined recombinant protein matrix designed to mimic native extracellular matrix (ECM) structure. They are composed of alternating ordered and disordered protein domains which convey the unique ability to be both liquid at low temperature and a solid, porous ECM at body temperature without the need for any additional crosslinking. The exact protein sequence can be manipulated by standard gene engineering, tailoring the properties of the resulting matrix to desired degrees of stiffness, porosity, and cell interaction.

Table 1 provides a summary of materials available for 3D cell culture.

TABLE 1
Comparison of the POPS, Matrigel, and PEG-based
hydrogels for three-dimensional cell culture.

| | | | | | Aberrant |
|---|---|---|---|---|---|
| Matrices | Major | Biochemical | Biomaterial | Scaffold | Cell |
| Ma | Components | Variation | Variation | Formation | Phenotypes |
| inSoma | Recombinant | None; | None; | Tunable | None |
| POPs | elastin- | recombinant | firmness is | temperature | observed |
| | derived | protein | consistent | based liquid- | |
| | peptides | | and uniform | solid | |
| | | | | transition | |
| Matrigel | Extracted | Many; | Firmness | Temperature- | Differentiation, |
| | basal | growth | varies ~2 | based liquid- | virus- |
| | membrane of | factors, ECM | fold within a | solid | induced |
| | murine | components | sample and | transition | changes |
| | sarcoma | can differ | batch-to- | | (LDHV) |
| | cells | by 10-fold | batch | | |
| | | between | | | |
| | | batches | | | |
| PEG-based | PEG of | none; | None; | Solid; | None |
| hydrogels | varying | chemically | firmness is | hydrolytic/ | observed |
| | lengths; | synthesized | consistent | proteolytic | to date |
| | Additions | | and uniform; | sites must be | |
| | include | | chemical | chemically | |
| | heparin, | | cross-linker | added to | |
| | cross-linkers | | required | allow | |
| | protease/ | | | remodeling | |
| | hydrolysis | | | | |
| | sites | | | | |

In embodiments, POPs support the in vitro viability and proliferation of both primary cells, stem cells and cell lines. In embodiments, the stem cells can include embryonic stem cells, adult stem cells, induced pluripotent stem cells, and the like. Moreover, the stem cells can include mesenchymal stem cells, hematopoietic stem cells, neural stem cells, epithelial stem cells, skin stem cells, and the like.

Moreover, POP formulations can be optimized for three-dimensional culture, and each formulations' support of organoid viability and proliferation can be evaluated. Further, transcriptomics can be deployed to determine if aberrant phenotypes are reduced compared to problematic industry standard matrices, such as Matrigel.

In embodiments, POPs can be used for organoid culture, including organoids based on neural, pancreatic, and liver tissues. Further, evaluations of POPs as a support matrix for in vivo tissue engineering approaches will be evaluated.

As introduced above, partially-ordered polymers function as a fully-defined three-dimensional culture matrix. The partially-ordered polymers of the present disclosure are made from recombinant proteins, allowing for reliable and consistent manufacturing methods. Moreover, the partially-ordered polymers of the present disclosure support cellular viability in culture. Therefore, their use in, inter alia, organoid and stem cell culture are demonstrated herein.

In embodiments, POPs can also be used for two-dimensional cell culture. For instance, the POPs can be applied to the surface of a cell culture dish. The temperature can be increased such that the POPs form a porous network and then cells can be cultured on the POPs. After sufficient cell culture and/or proliferation, the POPs can then be cooled until the porous network of the POPs resolubilize. At that time, the cells are lifted from the surface of the POPs as a result of the dissolution. Thus, cells can be grown and “passaged” without the introduction of enzymes that forcibly remove the cells from the culture surface.

In embodiments, POPs are recombinantly synthesized in E. coli by overexpression of a plasmid-borne gene that encodes the POPs. As a result, they can take advantage of the protein fermentation and scale-up process used already by the biopharmaceutical industry for inexpensive recombinant protein production. In certain embodiments, POPs are synthesized within yeast, a cell-free mammalian system, or other protein expression systems.

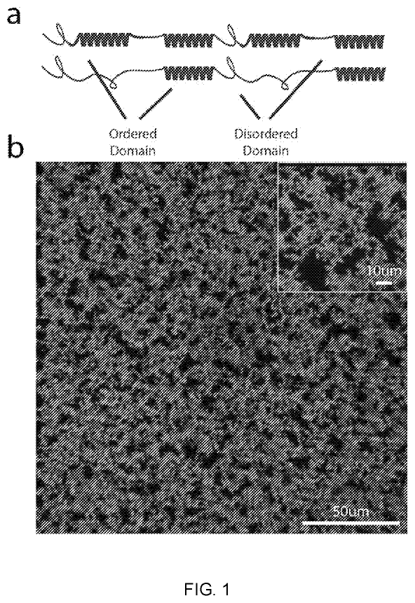

POPs consist of ordered, oligoalanine domains (e.g. A25)—that form perfect α-helices—that are periodically inserted into a disordered domains of unstructured elastin-like polypeptide (ELP) that is composed of typically 80-120 total repeats of a VPGXG pentapeptide (˜30-50 kDa; FIG. 1A). This unique combination of controlled order and disorder within a protein sequence allows exquisite control over POP handling and material properties. By altering the composition of these POPs and their segment organization, POPs can form mechanically stable, interpercolated networks (i.e. porous networks) with high surface to volume ratios and fractal dimensions similar to native elastin (FIG. 1B).

ELPs are comprised of repetitive polypeptides based on a repeated consensus [Val-Pro-Gly-Xaa-Gly](VPGXG) pentapeptide motif derived from the disordered regions of the extracellular matrix protein tropoelastin, wherein the guest residue “X” can be substituted with any amino acid except proline. One of the hallmarks of ELPs is their lower critical solution temperature (LCST) phase behavior, reversibly alternating between soluble and aggregated states at a critical threshold temperature, which may be tuned at the genetic level through variation of the guest residue. As a recombinant protein, production of pharmaceutical grade material in E. coli at a large scale is low-cost and simple, making them attractive for production of substantial quantities of protein required for their use as a cell substrate. Due to their biocompatibility and versatility, ELPs have been explored in a variety of biomedical applications including protein purification, injectable controlled release depots for drug delivery, as vehicles for tumor uptake of drugs and delivery of vaccines, and as matrices for cell and tissue engineering applications.

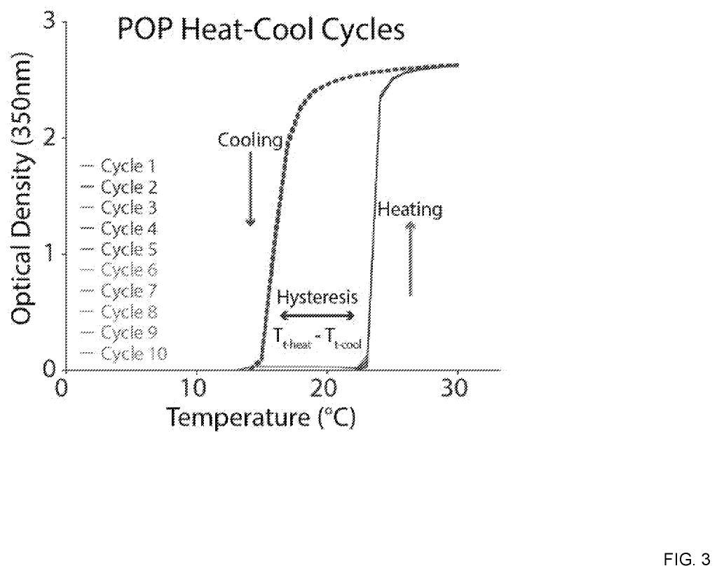

However, given their highly disordered nature, the three-dimensional structure of phase separated ELPs is that of a dense polymer coacervate with no internal microstructure. By systematically encoding ordered domains into ELPs, phase separation does not lead to dense coacervates, but rather to porous protein networks. In fact, despite the solid, microporous architecture, the networks remain reversible and thermally responsive. Unlike canonical ELPs, however, which have the same transition temperature (Tt) upon heating and cooling, POPs exhibit significant thermal hysteresis wherein the transition temperature of cooling (Tt-cooling) is up to 10° C. lower than the transition temperature of heating (Tt-heating). Thus, a POP coacervate is stable at a solution temperature between Tt-cooling and Tt-heating, which provides a wide operating range of temperature. In embodiments, both aggregation and dissolution occur on the time scale of seconds. Importantly, once fully solvated, polymers return to their original state and can be cyclically heated and cooled with no permanent alterations, as shown in FIG. 3. Polymers kept in an aggregated state for >24 hrs also show rapid re-solvation upon cooling.

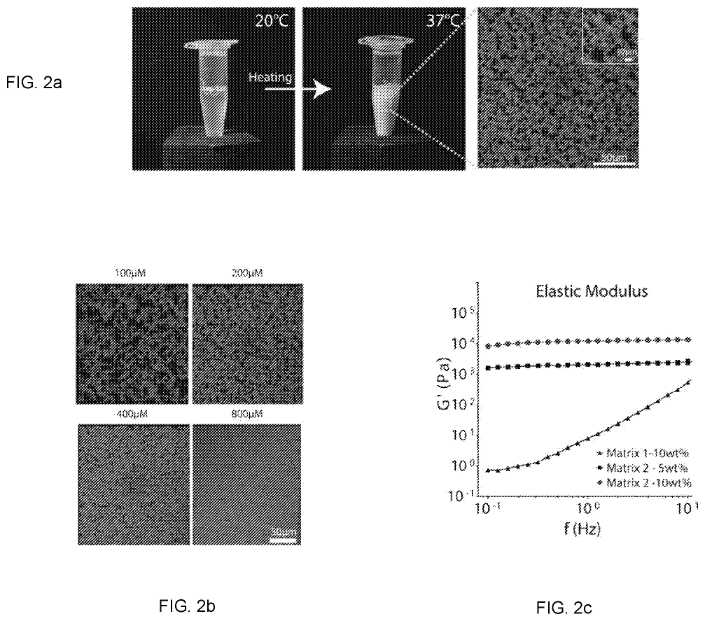

Phase separation of a POP solution upon increasing the temperature above its transition temperature (Tt) leads to formation of a physically crosslinked porous network that is reminiscent of crosslinked elastin networks (FIG. 2A). By tuning the composition and segment organization of POPs, mechanically stable, interpercolated networks with a high surface to volume ratio and a fractal dimension (D) of ˜1.7, similar to native elastin, can be designed. By modulating POP concentration, molecular weight, and density of physically crosslinking A25 segments, network pore sizes from 500 nm-20 μm (FIG. 2B), and mechanical stability, with elastic moduli (G′) ranging from 10 Pa to 10 kPa (FIG. 2C) can be controlled.

As noted above, despite the solid, microporous architecture, POP networks are thermally responsive. When heated, POPs will spontaneously form a network at a tunable Tt-heating and, unlike traditional thermally responsive materials, POP networks will dissolve at a separate, lower Tt-cooling. Thus, a POP coacervate is stable within the hysteretic range between Tt-cooling and Tt-heating, which provides an unusually wide operating range of temperatures. In reality, this means that networks formed in a cell incubator will not dissolve at room temperature, thus allowing easy (and cheaper) culture handling and media changes.

The following description of the Figures will focus on the ability to tailor the biomechanical and biochemical properties of POP networks for cell culture.

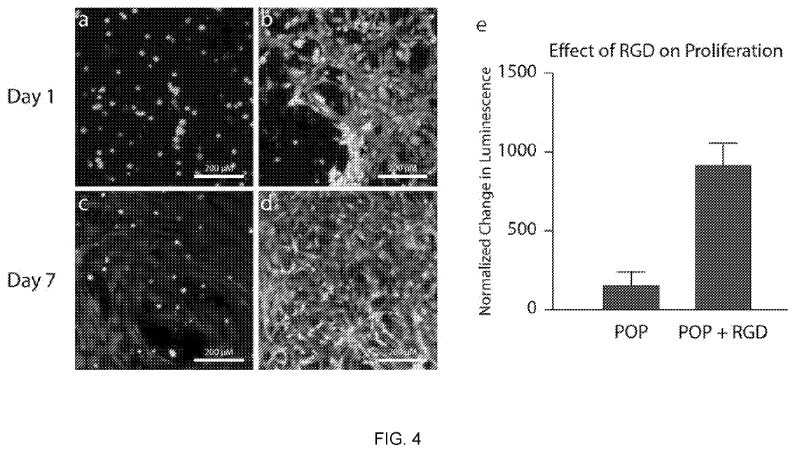

As an illustration of the ability to encode specific cell interactions within POP networks without altering their network formation ability, versions of POPs with cell-binding motifs (arginylglycylaspartic acid (RGDs)) spaced throughout the polymer in order to confer cell-adhesive behavior were designed. In embodiments, additional binding features can be added to the RGDs on either side of the RGD sequence. For instance, the additional binding features may be additional amino acids that affect a presentation of the RGD sequence. Bone marrow-derived human mesenchymal stem cells (hMSCs) were seeded with POP scaffolds with and without encoded RGD binding domains and incubated for 7 days (FIG. 4A-4E). While cells were viable without RGDs, their incorporation significantly increased adhesion, cell spreading, and proliferation. Similar methodology can be readily used to create POPs with poly-lysine, fibronectin and laminin domains.

### Polymer Library Design

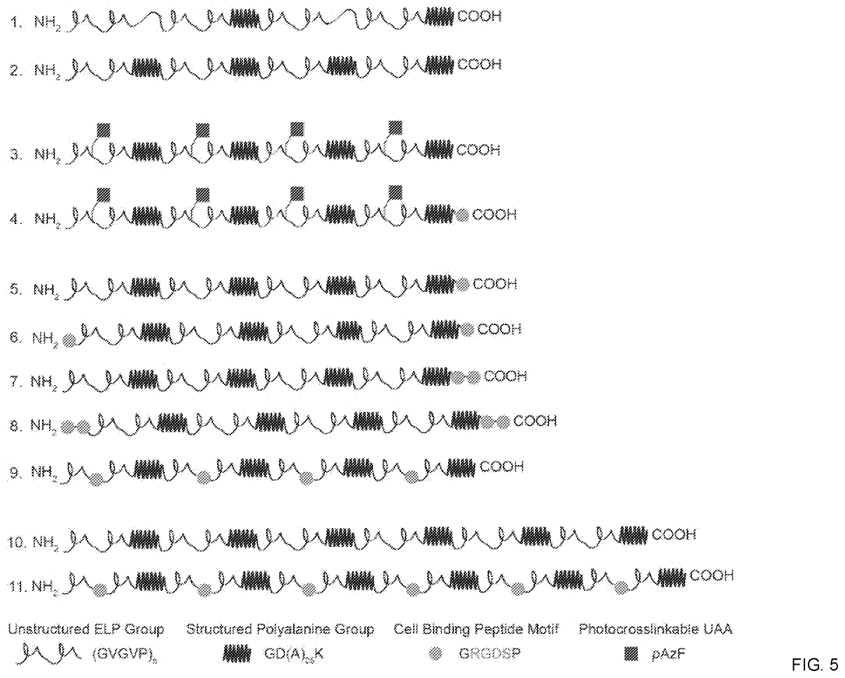

The general design of POPs is based on alternating groups of disordered ELP domains and ordered polyalanine domains (FIG. 1A). These sequences were previously optimized to include either 12.5 or 25% total helical content to insure proper network formation. This optimized POP was further modified with integrin binding domains to better control the interactions with cells cultured within the POP networks. These domains were added to the N and/or C terminus, or within the disordered ELP region of the polypeptide chains to determine the optimal conformation to promote cell adhesion and viability within the POP networks. A subset of POPs was further modified to be photo-crosslinked with ultraviolet (UV) light via the incorporation of an unnatural amino acid (UAA), paraazidophenylalanine (pAzF), within the disordered ELP regions of the polymer chain. A version of this photo-crosslinkable POP with a terminal cell-binding motif was created in order to confer cell-adhesive behavior for the crosslinked POP microparticles. Specific domain organization and the name use for each polymer are shown in FIG. 5.

### Development and Characterization of POP Microparticles

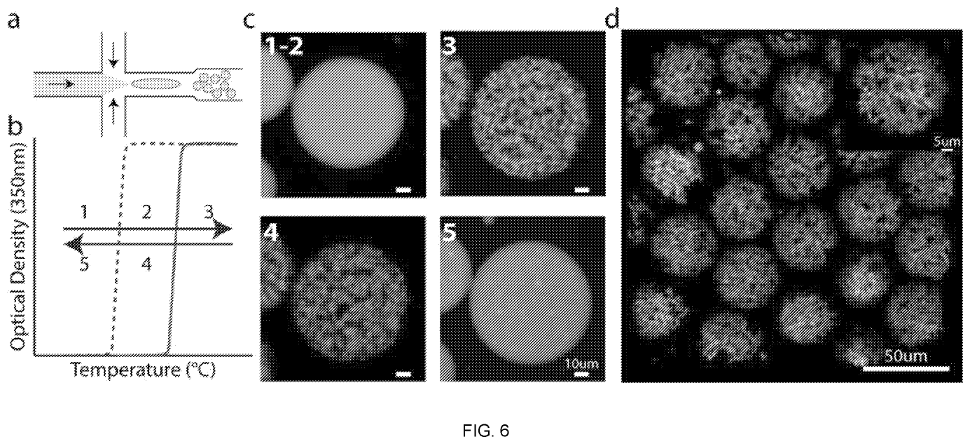

Even when encapsulated using water-in-oil microfluidics, POPs retrain the ability to form microscale fractal networks (FIG. 6). Heating the polymer droplets in these microenvironments produces stable microparticles with microarchitectures similar to those observed in bulk. Microparticle size is uniform and dependent the fluid flow rates used during formation. Subsequent cooling causes the polymer to fully dissolve back into solution.

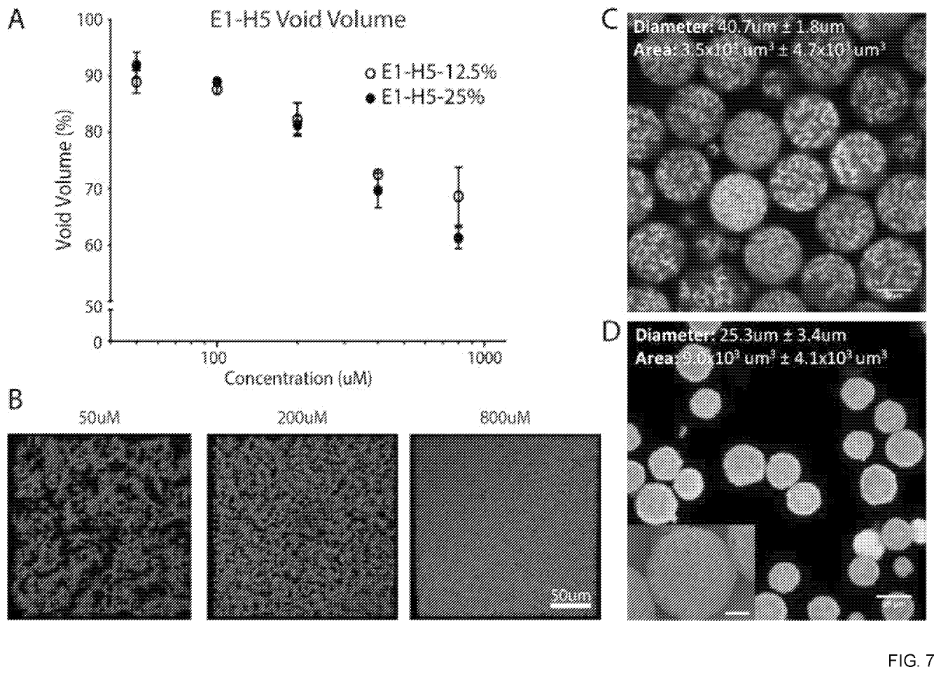

POP network porosity can be controlled by modulating polymer concentration. Using three-dimensional reconstructions from confocal microscopy, the effects of concentration on total void volume in bulk solution, defined as the non-protein rich phase of the network (FIG. 7A, 7B) were evaluated. Within a range of 50 μM (0.2 mg/ml) to 800 μM (2.6 mg/ml) for POP-80, the void volume can be tuned to be between 90% and 60% with no significant difference in void volume observed between the POPs with 12.5% and 25% helical content over all tested concentrations. The high degree of control that we have over porosity in bulk POP solutions translates well to microcarriers, with more concentrated POP solutions producing microcarriers with smaller pore sizes. Importantly, changing the composition of the ELP portion of POPs, which changes their thermal transition temperature, does not alter network structure, allowing independent control over network transition temperatures and porosity.

To permit POP microcarrier use in cell culture, an efficient method to remove the microcarriers from their production oil phase was developed. To this end, the microcarriers can be removed from the oil phase by (1) mixing with isobutanol, (2) centrifuging, and (3) resuspending in saline to remove all oil from microcarrier production (water-in-oil microfluidics) and to allow them to be stored and manipulated in aqueous environments. Initial tests demonstrated that the physical crosslinking between the polymers was insufficient to prevent shear deformation during extraction and processing. A variety of cross-linking techniques have been used before with ELPs, including amine-reactive crosslinking, chemical crosslinking, and UV crosslinking. The present disclosure introduces a biorthogonal crosslinking approach that genetically encodes a photoreactive unnatural amino acid, para-azidophenylalanine (pAzF) into the ELP backbone. This method eliminates the requirement for an extrinsic chemical crosslinker and is easier to optimize for cell release. Microparticles crosslinked and successfully extracted from the oil phase were smaller following extraction, though they remain porous, stable, and noninteractive several days after extraction (FIG. 7C, 7D). Polymers containing both 12.5% or 25% helical content were successfully extracted, though the 25% proved more stable and was, therefore, chosen as the base for all future modifications.

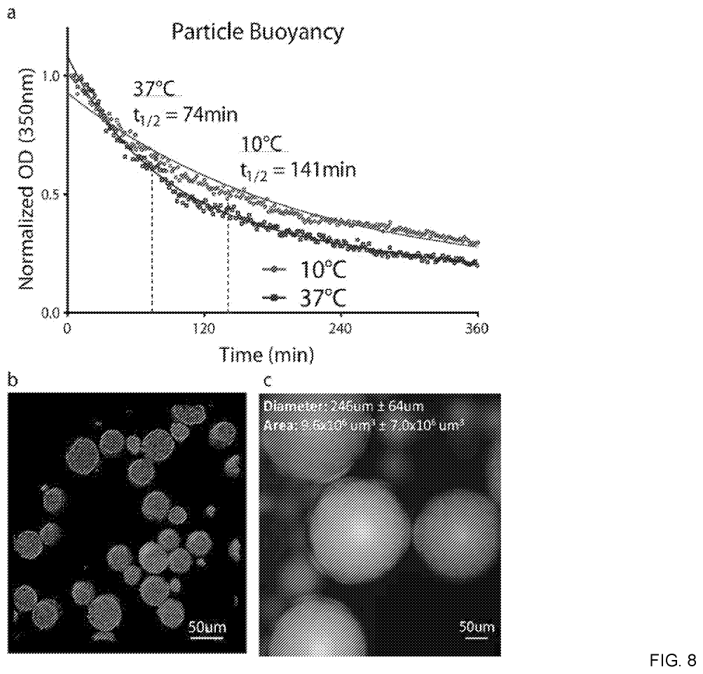

Buoyancy is an additional concern for microparticles as they need to maintain suspension to prevent settling during cell culture. To evaluate their buoyancy, POP microparticles with a mean diameter of 45 μm were suspended in PBS and the optical density of the solution was measured overtime to monitor particle settling. At storage conditions (less than their transition temperature, 10° C.) particles settled with a half-life of almost 2.5 hours, and at culture conditions (37° C.) they settled with a half-life of just over one hour (FIG. 8A). Three-dimensional reconstructions of these same microparticles using confocal microscopy reveal that, despite the slight size collapse they experience after extraction, they maintain a highly porous internal architecture that allows for the free movement and distribution of nutrients during culture (FIG. 8B). This method does allow for the reliable production of particles as small as 20 μm and as large as 250 μm in diameter (FIG. 8C). For particles 50 μm in diameter, 100k particles can be produced per hour at a density of ˜500k particles/ml.

### Cell Adhesive Properties of POP Networks

A primary advantage of POPs compared to other materials is that they provide a scaffold that is both natively biocompatible and immune tolerant. Thus, the scaffold can be tailored to the specific needs of an application by encoding specific interaction domains within the protein sequence. A common, and necessary, first approach to the design of biological scaffolds is the addition of cell-adhesive ligands, the most common of which is the RGD ligand. This peptide sequence initiates cell binding through integrin cell surface receptors, which promotes cell adhesion, spreading, and actin filament organization that is required for healthy cell proliferation on hydrogel surfaces. To promote cell binding and adhesion, POPs were therefore constructed with RGD sequences on the external N and/or C terminus of the chain or within the polypeptide chain. Given the number of sequence variables tested and the processing time limitations of POP microparticles, cell attachment in traditional cell culture plates with POPs acting as a 3D scaffold was optimized. The viability of POP matrices was tested with human bone-marrow or adipose-derived mesenchymal stem cells. Specifically, the capacity for cells to adhere, spread, and proliferate over and within the POP networks was evaluated. In order to test the viability of POPs as an alternative matrix material, human bone marrow-derived mesenchymal stem cells (hMSCs) were embedded within 250 μM POP networks containing a single terminal RGD sequence. Live/Dead assays were performed at days 0 and 8 using a concentration of 4 μM ethidium homodimer-1 to denote dead cells and 2 μM Calcein AM to denote live cells (FIG. 9). Cells were embedded at a high density (2000 cells/L POP) and allowed to adhere for 4 hours prior to an initial media change and live/dead stain at day 0. Although cells embedded within unmodified POP networks remained associated with the POP network at day 0 (FIG. 9A), a much greater retention of cells overall was detected within the RGD POP networks (FIG. 9D). After 8 days in culture, many cells had detached from the unmodified POP and migrated away from the network onto the tissue culture plate underneath (FIG. 9B, 9C)—however, cells embedded within the RGD POP remained closely associated with the network and formed a compacted cell sheet (FIG. 9E, 9F), suggesting extracellular matrix formation that may impact the capacity of cell removal from a POP network after cooling.

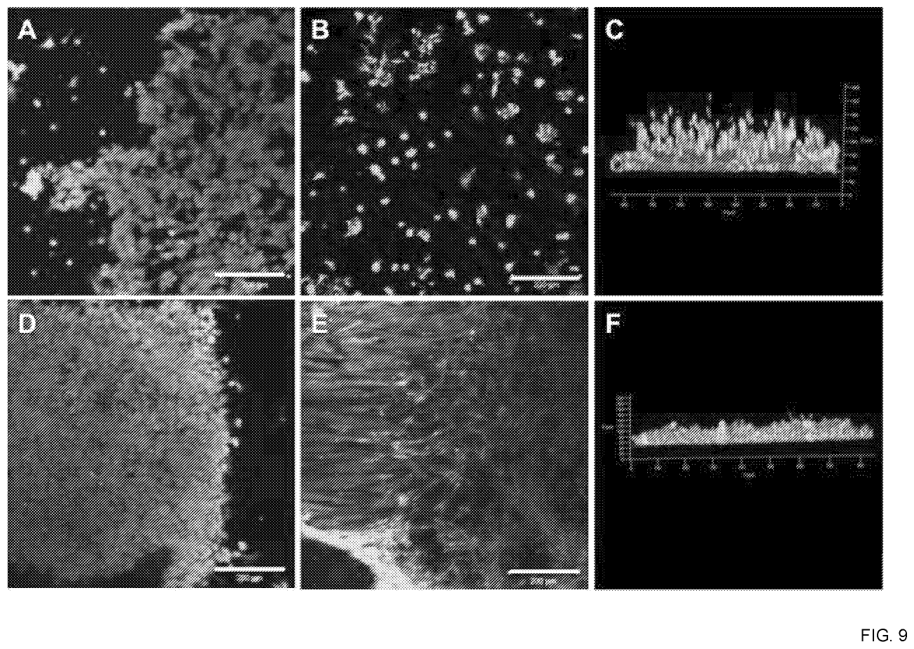

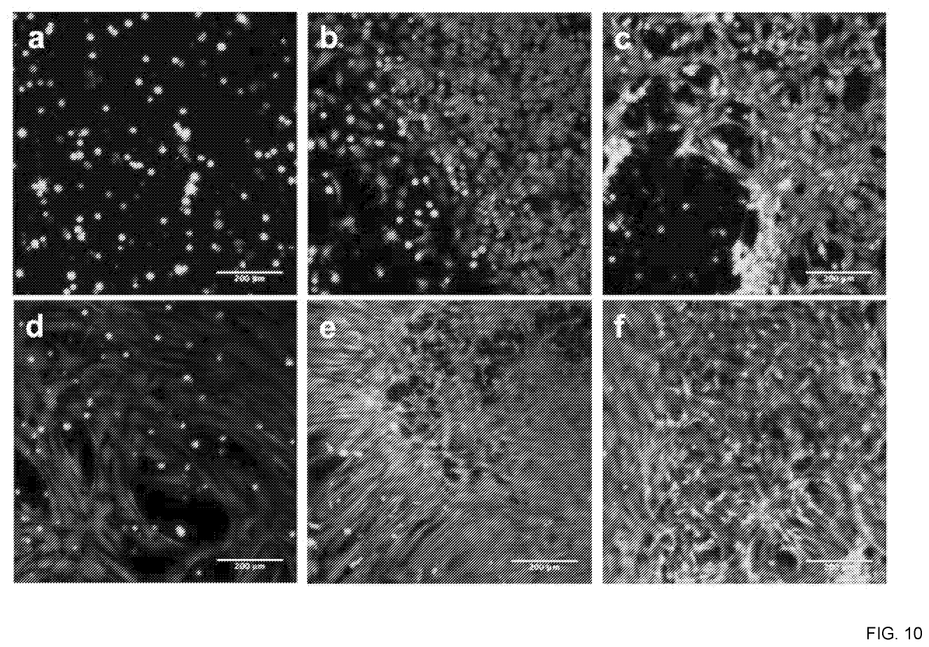

Due to the cell behavior detected in this experiment, whether a reduced RGD concentration (controlled by controlling the ratio of unmodified to modified POP in solution) in the network would still promote cell attachment and retention within the POP network was assessed. Cell adhesion and spreading over 7 days with hMSCs embedded within POP-80, RGD-POP-80A, and a 1:1 mixture of POP-80:RGD-POP-80A was compared. Reducing the RGD concentration by 50% was found to reduce the initial cell spreading on the POP networks on day 0 of culture compared to RGD-POP-80A (FIG. 10B), and after 7 days, cell migration away from the bulk POP networks (FIG. 10E) was observed. From this experiment, it was concluded that at least one RGD peptide was required to be present on all of the polymer chains within the POP network to ensure that cells remain associated with the POP networks in culture—however, more optimization was required from this point to ensure that cells would proliferate within the network without excessive extracellular matrix formation. One critical aspect to note is that cell culture media comprised of 20% fetal bovine serum (FBS) was used in these preliminary experiments.

Higher concentrations of FBS in the presence of RGD-functionalized biomaterials will promote greater adsorption of serum proteins onto the biomaterial surfaces, which can alter the cell integrin interactions with the RGD ligands present. It is likely that some of the adhesive behavior observed in these preliminary experiments was due in part to the presence of serum proteins that promoted cell association with the POP networks. To ensure that the cell adhesive behavior is due specifically to the material properties of the POP networks to ensure their viability and compatibility within a broad variety of in vitro cell culture applications, the following experiments were performed with growth media supplemented with 2-7% FBS, as noted in the methods.

As extracted POP microparticles are UV crosslinked prior to extraction, an RGD group was fused with the C-terminus of UV-crosslinkable POP (UV-RGD-POP) and the capacity for hMSCs to infiltrate through the POP matrix was evaluated. The impact of polymer concentration, and therefore porosity, on the ability of hMSCs to bind to the matrix was also tested. POP concentrations of 250 μM and 750 μM were crosslinked for 1 minute with a 312 nm UV lamp (previously determined to be sufficient for complete polymer crosslinking). hMSCs were plated over POPs at a concentration of 333 cells/uL POP and allowed to adhere for 24 hours. Cells remained adhered to POPs after 24 hours, although 750 μM POP retained an approximately 70% greater average number of cells compared to 250 μM POP. Cells adhered to the 250 μM POP were able to infiltrate the POP network, despite the lower number of cells adhered at 24 hours.

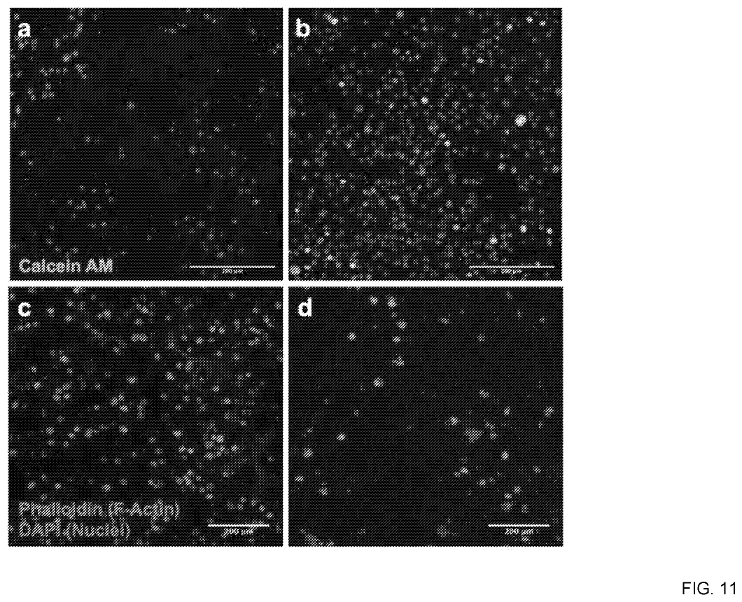

At a higher concentration, the cells adhered were concentrated on the surface, suggesting that they were not as capable of penetrating through the cross-linked POP networks at a higher concentration. This is not unusual with crosslinked hydrogels. In fact, previously, groups have incorporated protease sites within polymer chains of hydrogels in order to promote degradation of synthetic matrices by cell secreted MMPs. In the present disclosure, cells were cultured on the surface of both the RGD-POP-80A and the crosslinked UV-RGD-POP for 1 week (FIG. 11C, 11D). Both polymers were diluted to a concentration of 750 μM and fully crosslinked. The UV-RGD-POP was crosslinked with 1 minute of UV exposure, and the RGD-POP-80A was chemically crosslinked with tetrakis(hydroxymethyl) phosphonium chloride (THPC). hMSCs remained adhered on the surface of both POP compositions for 1 week, however, cells remained fairly rounded with minimal actin polymerization. These results contrast those shown in FIG. 9 and FIG. 10 with RGD POPs due to the reduced FBS concentration present within the growth media, suggesting that additional cues may be necessary for cells to spread, infiltrate, and proliferate within the polymer networks.

### Cell Adhesive Properties of POP Microparticles

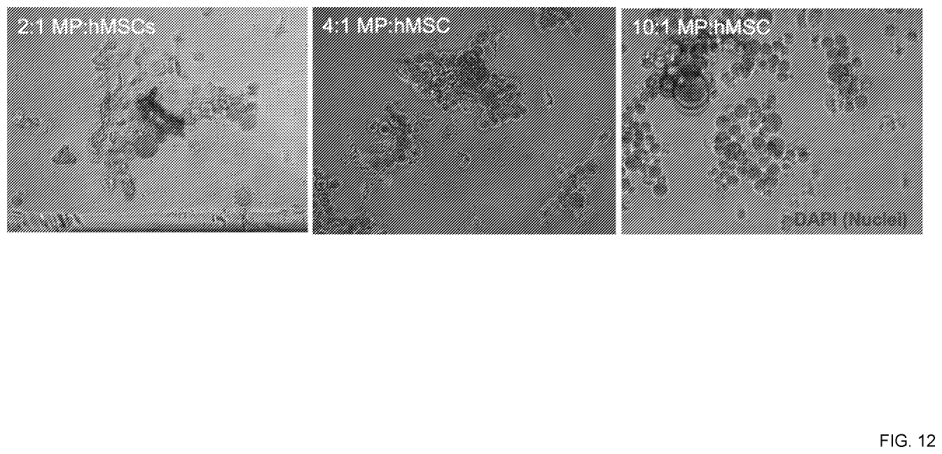

After determining that cells were able to adhere to the UV-RGD-POP-80 fusions, the behavior of hMSCs within microparticles comprised of this material was tested (FIG. 12). A microfluidic droplet generating chip was used to create water-in-oil emulsion droplets that were then able to be extracted from the oil suspension with isobutanol and UV-crosslinked for stability. Cells were cultured on POP microparticles over 5 days to determine an optimal ratio of microparticles to cells. 2:1, 4:1, and 10:1 microparticles to hMSCs were evaluated. Microparticle suspensions were resuspended in hMSC media and cultured on a rocker for 5 days to simulate the dynamic flow conditions of rotating bioreactor culture. After 5 days, particle and cell solutions were fixed with 4% PFA and stained with DAPI. Cells were found to preferentially adhere to the microparticles, however, the size of the particles was substantially smaller than the cells, and most importantly, particles tend to aggregate at cell culture temperatures since the transition temperature of the POP is substantially lower than 37° C. From these experiments and those using POPs as a 3D cell scaffold, POPs with (a) a higher transition temperature and (b) greater cell binding potential were needed to achieve the goal of creating discrete microcarriers at 37° C.

Further Optimization of Cell Binding Domains and Tt for hMSCs

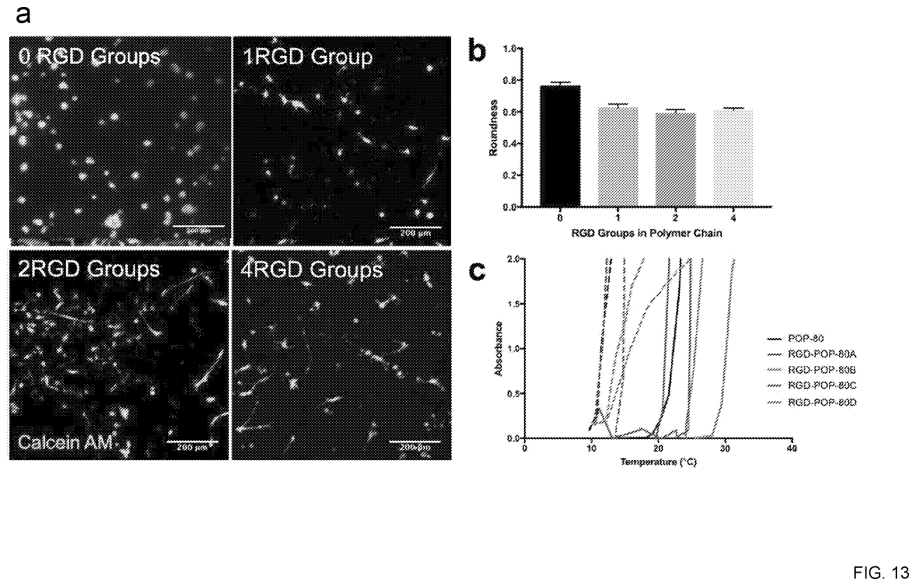

Results with a single RGD on each polymer suggested that adding more cell binding motifs to the polymer chain would help facilitate greater cell adhesion, spreading, actin polymerization, and proliferation. In response, three additional derivatives of POP-80 were created: 1) one RGD group on both the N and C terminus, 2) two RGD groups on the C-terminus, and 3) two RGD groups on both the N and C terminus. The addition of more RGD groups to the ends of the polymer chains was found to not substantially increase cell spreading compared to one RGD group, however, it did help to increase cell density and migration through the POP networks (FIG. 13A).

The presence of RGD groups reduced the roundness of adhered cells compared to POP without RGD, demonstrating that cells were spreading more readily. Bone-marrow derived hMSCs also migrated more deeply into the POP matrix after 2 days in culture when seeded on the surface of 500 μM POP networks. Given the charge present on the RGD peptides, their further addition was anticipated to not only improve cell spreading but to serve to increase the Tt of the POPs. As determined by optical density measurements, the Tt of the polymers increased with a greater number of RGD groups (FIG. 13C). The improved range is still within a temperature range appropriate for cell culture and release but will be beneficial in avoiding microparticle aggregation at 37° C.

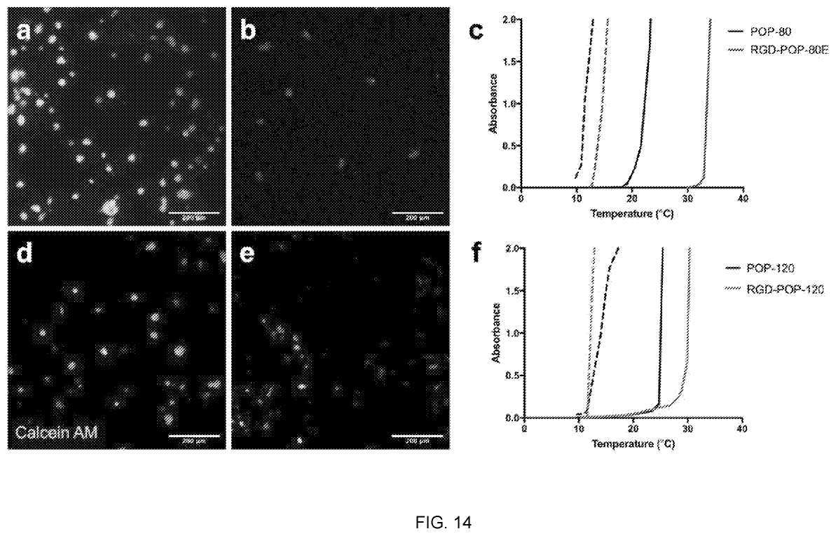

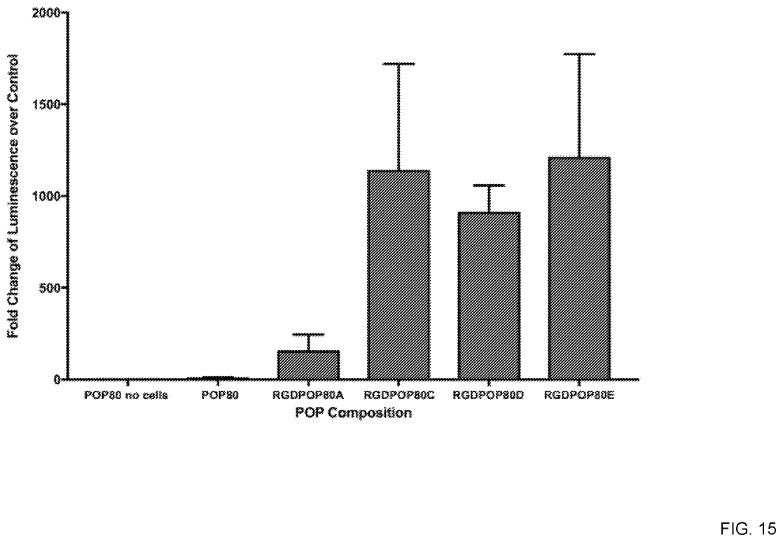

Noting that increasing RGD motif density on the POP networks enhanced cell spreading and migration, whether having RGD groups spaced periodically within the polypeptide chain would promote even greater cell proliferation was assessed. Two additional compositions of POPs with RGD groups were created and incorporated within the polypeptide chain, referred to herein as RGD-POP-80E and RGD-POP-120, which included 4 and 6 RGD groups per chain, respectively. Optical density measurements demonstrated that including the RGD groups within the polymer chain further increased the transition temperature compared to the unmodified POPs (FIG. 14).

Initially, adhesion of 10,000 hMSCs onto the polymer chains at a concentration of 250 μM was tested, similar to the other assays performed. Substantially lower cell adhesion than anticipated at 48 hours was found, suggesting that the POPs with RGD sequences within the polymer chain did not lend themselves to increased RGD availability.

Lastly, a commercial cell viability and proliferation assay was used to obtain quantifiable data regarding the proliferative capacity of cells on the POP networks. POP solutions at a density of 250 μM were plated within 387 well plates, allowed to form networks, and 25,000 adipose-derived hMSCs were seeded over the networks and allowed to incubate for 3 days (FIG. 12). Adipose-derived hMSCs were used to confirm the results previously obtained with bone marrow-derived hMSCs. Due to the capacity for cells to migrate away from POPs, particularly in compositions without RGD groups, POPs with MSCs were transferred from their initial plates to wells of 96-well plates filled with media. Viable cells were assessed using the CellTiter-Glo Luminescent Cell Viability Assay (Promega), which detects metabolically active cells based on the quantitation of ATP present within the cells. Overall, the results from the Cell Titer Glo assay suggest that a greater amount of RGD groups present within the polymer chain allows for greater viable cell retention within the POP network, with no clear difference between the POPs with RGD groups within the chain (RGD-POP-80E) and POPs with greater than 2 RGD groups on the terminal ends of the chains (RGD-POP-80C or RGD-POP-80D).

### POPs as Crosslinkable Matrix Substitutes

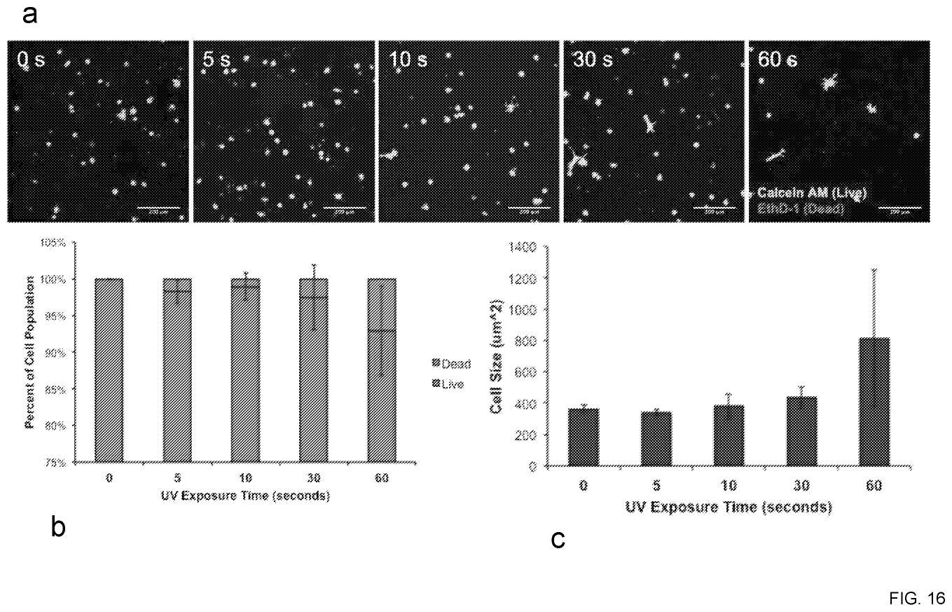

The capacity of the UV-RGD-POP-80 as a matrix material wherein cells could be embedded prior to UV crosslinking was explored as a potential alternative approach to microparticle culture. To this end, 5,000 hMSCs where embedded in 30 μL of 250 μM POP, followed by cross-linking the networks with UV exposure for 0-60 seconds (FIG. 16A-C). Cells tolerated UV exposure after embedding within the POP network for up to 30 seconds with no change in their viability or cell spreading capacity, however, exposure after 60 seconds promoted cell death and apoptosis of cells within the POP network. This study demonstrated that UV-crosslinkable POPs allow for cell viability after UV exposure at levels amenable for cell encapsulation studies.

Recovery of Cell from POP Networks with Cooling

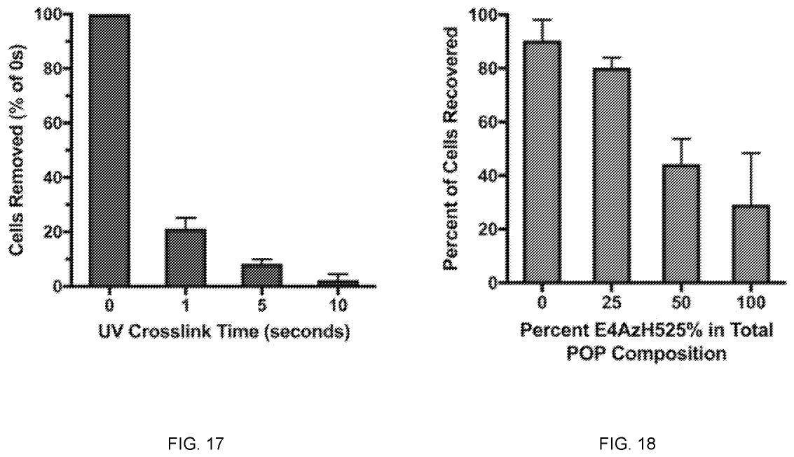

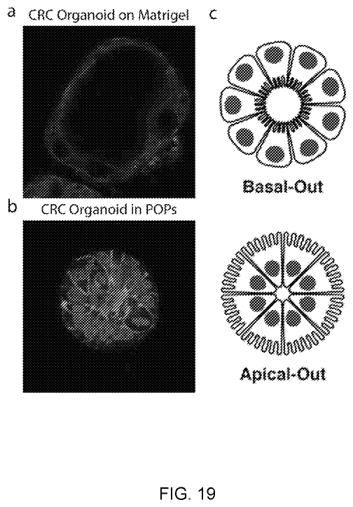

Whether cells would be viable and capable of re-adhering to tissue culture plates after extraction from cross-linked POPs was assessed. Cell suspensions in hMSC growth medium were embedded within POPs at a density of 170 cells/μL of POP, with POP used at a concentration of 250 μM, and maintained in hMSC growth medium within a 384 well plate. After 2 days, after which cells were able to adhere and spread on the matrix, the entire POP solution was removed from the well and transferred to microcentrifuge tubes in 200 μL DPBS. Solutions were cooled to 4° C. for 5 minutes to allow POPs to transition to their liquid state, and then centrifuged at 300×g for 5 minutes at 4° C. The remaining cell pellets were resuspended in hMSC media and plated overnight, fixed, and stained with DAPI. UV crosslinking of POPs for 1 second reduced the number of viable cells adhered to 80% that of un-crosslinked POPs (FIG. 17). This study demonstrated that recovery of cells from cross-linked bulk POP networks is feasible. However, optimization of the polymer composition to promote greater cell attachment and infiltration may result in greater numbers of cells being released from more highly crosslinked networks. Four sites per polymer chain that can be crosslinked with UV exposure were found to substantially reduce the removal of cells after crosslinking for 1 second, with cell release dropping as the rate decreases. To determine the cell removal rate from the POP network after UV exposure. Four dilutions of UV-RGD-POP were created with 75% or 50% POP without UV crosslinking sites (RGD-POP-80A). 30 μL bulk POP networks were created at a concentration of 500 μM POP in 1×PBS, allowed to form coacervates, and then UV cross-linked for 2 seconds. 10,000 bone marrow-derived hMSCs were seeded on the surface in growth medium and allowed to adhere overnight. POPs were cooled for 10 minutes, and then resuspended in a total volume of 200 μL media. A hemocytometer was used to determine the total cell count within each group. Overall, lightly crosslinking a mixture containing 25% photo-crosslinkable POP, or an average of 1 cross-linking site per polymer chain, yielded the greatest cell release, comparable to no crosslinks per chain (FIG. 18). Reducing the amount of crosslinked matrix by 75% promoted cell removal levels comparable to that of POPs with no crosslinking, suggesting that cells require more flexibility to move throughout the polymer chains.

### Optimizing POP Composition to Promote Cell Proliferation

Overall, most cell lines prefer polymer surfaces with moderate hydrophilicity and demonstrate poor adhesion and proliferation on polymer surfaces that are highly hydrophilic or hydrophobic. ELP-based hydrogels created with more polar amino acids in the guest residue space, such as isoleucine, have demonstrated high cell adhesion, spreading, and actin polymerization.

### Supplementary Methods

### Synthesis of Polymer Genes

All POPs were cloned as previously described. All polymers were cloned into a modified pet24 vector using a process known as recursive directional ligation by plasmid reconstruction (PRe-RDL). Single-stranded oligomers encoding the desired sequences were annealed into cassettes with CC and GG overhangs, allowing concatemerization and ligation into the pet24 vector. This was used to create a library of ELP and polyalanine cassettes which could be strung together through multiple cycles of PRe-RDL to form the final POP compositions. Plasmids were transfected into chemically competent EB5a cells for cloning and BL21(DE3) cells for protein expression.

### Expression and Purification of POPs

For protein expression, 50 mL starter cultures were grown overnight from −80 C glycerol stocks or freshly transformed BL21(DE3) cells. Cells were then used along with 1 mL of 45 mg/mL kanamycin to inoculate 1 L of 2xyt media. Cells were shaken at 200 rpm for 6 hours at 25 C before induction of protein expression with 1 μM isopropyl b-D-1-thiogalactopyranoside (IPTG) was added to the flask. After induction, temperature was lowered to 16 C and cultures were continued to be shaken at 200 rpm overnight, which was necessary to prevent the formation of truncation products at ELP-polyalanine junctions. Cells were pelleted and resuspended in 10 mL of 1× phosphate buffered saline (PBS) for every 1 L of culture grown. Pulse sonication on ice with a total active time of 3 minutes was used to lyse cells. Cell lysates were treated with 10% polyethyleneimine (PEI) (2 mL/L culture) to remove contaminating DNA and centrifuged at 14000 rpm for 10 minutes at 4 C. Polymer was purified from the resulting soluble fraction using a modified version of inverse transition cycling. The fraction was heated to 65 C or until phase separation was observed. For more hydrophilic polymers, this often required the addition of 1-2 μM NaCl to depress the transition temperature. Once aggregated, the polymer solutions were centrifuged at 14000 rpm for 10 minutes at 35 C and the resulting pellet was resuspended in 5-10 mL PBS. The heating and cooling centrifugation cycles were repeated 2-3 more times until a purity of 95% was achieved, as analyzed by sodium dodecyl sulfate polyacrylamide gel electrophoresis (SDS-PAGE). Purified polymers were dialyzed at 4 C with frequent water changes for 2 days or buffer exchanged using centrifuge filters with a 10000 MWCO (Amicon) and lyophilized for storage.

### Cell Culture

Human bone marrow derived mesenchymal stem cells and human adipose-derived mesenchymal stem cells were purchased from ATCC and cultured according to the manufacturer's specifications. Bone-marrow derived hMSCs were cultured using the ATCC mesenchymal stem cell growth kit for bone-marrow derived MSCs, which contained a basal medium supplemented with 5 ng/mL rh FGF basic, 15 ng/mL rh IGF-1, 7% fetal bovine serum, and 2.4 mM L-alanyl-Lglutamine.

Adipose-derived hMSCs were cultured using the ATCC mesenchymal stem cell growth kit for Adipose and Umbilical cord derived MSCs, which included a basal medium supplemented with 5 ng/mL rh FGF basic, 5 ng/mL rh FGF acidic, 5 ng/mL rh EGF, 2% fetal bovine serum, and 2.4 mM L-alanyl-L-glutamine. 1% Pen/Strep was added to all culture media. Cells were used between passage 3-6 for all experiments.

The present disclosure describes porous microparticles from POP networks using water-in-oil microfluidics. These particles are monodisperse and buoyant. Cell-binding RGD peptides can be fused to the polymer chains. A photo-crosslinkable POP with cell-binding capability can be generated and cell removal from cross-linked POP networks is achievable. Overall, this work demonstrates the capacity for POPs to be optimized as either 3D matrix substitutes for cell culture either as bulk networks or porous microcarriers.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

### Numbered Embodiments of the Invention

Notwithstanding the appended claims, the disclosure sets forth the following numbered embodiments:
(1) A composition for three-dimensional cell and/or organoid culture, comprising a phase-changing matrix comprising a partially ordered polypeptide, and a plurality of cells encapsulated in the matrix.
(2) The composition of (1), wherein the plurality of cells comprises one or more cell types.
(3) The composition of either (1) or (2), wherein the one or more cell types comprises at least one cell type selected from the group consisting of: bone-marrow derived mesenchymal stem cells and adipose-derived mesenchymal stem cells.
(4) The composition of either (1) or (2), wherein the one or more cell types comprises mesenchymal stem cells.
(5) The composition of any one of the preceding claims, wherein the partially ordered polypeptide comprises at least one chemically-interactive sequence.
(6) The composition of (5), wherein the at least one chemically-interactive sequence is an arginylglycylaspartic acid (RGD) sequence.
(7) The composition of (5), wherein the at least one chemically-interactive sequence is incorporated from the N terminus and/or the C terminus of the partially ordered polypeptide.
(8) The composition of (5), wherein the at least one chemically-interactive sequence is incorporated between the N terminus and the C terminus of the partially ordered polypeptide.
(9) The composition of any one of the preceding claims, wherein the matrix forms a porous network when heated above a transition temperature.
(10) The composition of (8), wherein a concentration of the partially ordered polypeptide within the porous network is between about 0.2 mg/ml and about 300 mg/ml.
(11) The composition of (8), wherein a void volume of the porous network is between about 60% and about 90%.
(12) The composition of (8), wherein a void volume of the porous network is substantially unchanged with the helical content of the partially ordered polypeptide is varied between about 12.5% and about 50%.
(13) The composition of any one of the preceding claims, wherein the partially-ordered polypeptide comprises a plurality of structured domains, and a plurality of disordered domains.
(14) The composition of (12), wherein the partially ordered polypeptide is modified to encode a photoreactive unnatural amino acid into at least one of the plurality of unstructured domains.
(15) The composition of (12), wherein at least one of the plurality of disordered domains comprises a plurality of an amino acid sequence of (GXGVP)ₙ, wherein X is any amino acid except proline and n is an integer greater than or equal to 1, and the structured domain comprises a polyalanine domain.
(16) The composition of (13), wherein the disordered domain comprises a plurality of an amino acid sequence of (GXGVP)ₙ, wherein X is Val, or Ala, or a mixture of Ala and Val, and wherein n is an integer from 1 to 50.
(17) The composition of (16), wherein X is an alternating iteration of Ala and Val in a ratio from 10:1 to 1:10 (Ala:Val).
(18) The composition of (17), wherein X is an alternating iteration of Ala and Val in a ratio of 1:1 or 1:4.
(19) The composition of (15), wherein the polyalanine domain comprises (Ala)ₘ, wherein m is an integer from 5 to 50.
(20) The composition of (15), wherein the polyalanine domain comprises one or more of (A)₂₅, K(A)₂₅K, D(A)₂₅K, GD(A₂₅)K, or GK(A₂₅)K.
(21) The composition of (15), wherein the partially-ordered polypeptide comprises [(GXGVP)ₙ-GX¹(A)₂₅X¹]ₘ, where X is A or V; X¹ is K or D, n is an integer from 10 to 20, and m is an integer from 4 to 8.
(22) The composition of (15), wherein the partially-ordered polypeptide comprises one or more of M[(GVGVP)₁₅-GD(A₂₅)K]₆-GWP, M[(GVGVP)₁₅-GD(A₂₅)K]₄-GWP, M[(GVGVP)₁₅-GK(A₂₅)K]₆-GWP, M[(GVGVP)₁₅-GK(A₂₅)K]₄-GWP, M[(G[A₁:V₁]GVP)₁₆-GD(A₂₅)K]₆-GWP, M[(G[A₁:V₁]GVP)₁₆-GD(A₂₅)K]₄-GWP, M[(G[V₄:A₁]GVP)₁₅-GD(A₂₅)K]₆-GWP, or M[(G[V₄:A₁]GVP)₁₅-GD(A₂₅)K]₄-GWP.
(23) The composition of claim (15), wherein the partially-ordered polypeptide comprises one or more of M[(GVGVP)₁₅-GD(A₂₅)K]₆-GWP, or M[(G[V₄:A₁]GVP)₁₅-GD(A₂₅)K]₆-GWP.
(24) The composition of any one of the preceding claims, wherein the matrix has a transition temperature of heating (Tt-heating) and a transition temperature of cooling (Tt-cooling).
(25) The composition of (24), wherein the transition temperature of cooling (Tt-cooling) is concentration-independent.
(26) The composition of (24), wherein the transition temperature of heating (Tt-heating) and the transition temperature of cooling (Tt-cooling) range from about 10° C. to about 45° C.
(27) The composition of (24), wherein the matrix forms a solid aggregate above the Tt-heating.
(28) The composition of (27), wherein the solid aggregate resolubilizes when cooled to below the Tt-cooling.
(29) The composition of (27), wherein the solid aggregate is a stable three-dimensional matrix.
(30) The composition of (27), wherein the solid aggregate comprises a plurality of micropores.
(31) The composition of (30), wherein the plurality of micropores range in size from about 1 μm to about 150 pm.
(32) The composition of any one of the preceding claims, wherein the composition comprises between about 200 pM and about 2 mM of the matrix.
(33) The composition of any one of the preceding claims, wherein the composition comprises the matrix and the plurality of cells in a ratio ranging from about 1:9 to about 9:1.
(34) The composition of any one of the preceding claims, wherein the composition comprises the matrix and the plurality of cells in a ratio ranging from about 1:3 to about 3:1.
(35) The composition of any one of the preceding claims, wherein the composition comprises the matrix and the plurality of cells in a ratio of about 1:1.
(36) The composition of any one of the preceding claims, wherein the composition is a shapeable liquid or semisolid.
(37) The composition of any one of the preceding claims, wherein the composition is injectable or implantable.
(38) The composition of any one of the preceding claims, wherein the composition is shapeable or moldable into 2- or 3-dimensional shapes, areas, or volumes.
(39) The composition of any one of the preceding claims, wherein the matrix permits cell infiltration and vascularization.
(40) A method of three-dimensional cell culture, comprising mixing, at a first temperature, a plurality of cells with a phase-changing matrix comprising a partially ordered polypeptide, heating the mixture to a second temperature that is higher than the first temperature, wherein the phase-changing matrix forms a porous network when heated to the second temperature, the porous network having a three-dimensional structure comprising the phase-changing matrix and the plurality of cells, the plurality of cells being encapsulated therein, and culturing the solid aggregate at physiological conditions.
(41) The method of (40), wherein the plurality of cells comprises one or more cell types.
(42) The method of (41), wherein the one or more cell types comprises at least one cell type selected from the group consisting of: bone-marrow derived mesenchymal stem cells and adipose-derived mesenchymal stem cells.
(43) The method of (41), wherein the one or more cell types comprises mesenchymal stem cells.
(44) The method of any one of (40) to (43), wherein a concentration of the partially ordered polypeptide within the porous network is between about 0.2 mg/ml and about 300 mg/ml.
(45) The method of any one of (40) to (44), wherein a void volume of the porous network is between about 60% and about 90%.
(46) The method of any one of (40) to (45), wherein a void volume of the porous network is substantially unchanged with the helical content of the partially ordered polypeptide is varied between about 12.5% and about 50%.
(47) The method of any one of (40) to (46), wherein the partially-ordered polypeptide comprises a plurality of structured domains, and a plurality of disordered domains.
(48) The method of (47), wherein at least one of the plurality of disordered domains comprises a plurality of an amino acid sequence of (GXGVP)ₙ, wherein X is any amino acid except proline and n is an integer greater than or equal to 1, and the structured domain comprises a polyalanine domain.
(49) A method of extracting a plurality of cells from three-dimensional cell culture, comprising cooling, to a first temperature, a mixture of the plurality of cells and a phase-changing matrix comprising a partially ordered polypeptide, the phase-changing matrix solubilizing at the first temperature, and extracting the plurality of cells from the solubilized phase-changing matrix.
(50) A method for tissue replacement and/or augmentation, comprising mixing, at a first temperature, a plurality of cells with a phase-changing matrix comprising a partially ordered polypeptide, heating the mixture to a second temperature that is higher than the first temperature, wherein the phase-changing matrix forms a porous network when heated to the second temperature, the porous network having a three-dimensional structure comprising the phase-changing matrix and the plurality of cells, the plurality of cells being encapsulated therein, and implanting the solid aggregate into a subject.
(51) A method for tissue replacement and/or augmentation, comprising mixing, at a first temperature, a plurality of cells with a phase-changing matrix comprising a partially ordered polypeptide, heating the mixture to a second temperature that is higher than the first temperature, wherein the phase-changing matrix forms a porous network when heated to the second temperature, the porous network having a three-dimensional structure comprising the phase-changing matrix and the plurality of cells, the plurality of cells being encapsulated therein, culturing the solid aggregate at physiological conditions, and implanting the solid aggregate into a subject at a target region.
(52) A method for tissue replacement and/or augmentation, comprising mixing, at a first temperature, a plurality of cells with a phase-changing matrix comprising a partially ordered polypeptide, and injecting the mixture into a subject at a target region, the phase-changing matrix forming a porous network when heated at least to a second temperature greater than the first temperature, the second temperature being less than or equal to body temperature, wherein the porous network has a three-dimensional structure comprising the phase-changing matrix and the plurality of cells, the plurality of cells being encapsulated therein.
(53) The composition of any one of (1) to (39), wherein the plurality of cells encapsulated in the matrix form at least one organoid.
(54) The method of any one of (40) to (48), wherein the cultured plurality of cells forms an organoid.
(55) A method of two-dimensional cell culture, comprising depositing, at a first temperature, a phase-changing matrix comprising a partially ordered polypeptide onto a culture surface, heating the deposited phase-changing matrix to a second temperature, wherein the phase-changing matrix forms a porous network when heated to the second temperature, seeding a plurality of cells onto a surface of the porous network, and culturing the plurality of cells.
(56) The method of (55), further comprising cooling the phase-changing matrix to the first temperature such that the phase-changing matrix solubilizes and extracting the cultured plurality of cells from the solubilized phase-changing matrix.

1. A composition for two-dimensional cell culture, three-dimensional cell culture, and/or organoid culture, comprising:
a phase-changing matrix comprising a partially ordered polypeptide; and
a plurality of cells encapsulated in the matrix.

2. The composition of claim 1, wherein the plurality of cells comprises one or more cell types.

3. The composition of claim 2, wherein the one or more cell types comprises at least one cell type selected from the group consisting of: bone-marrow derived mesenchymal stem cells and adipose-derived mesenchymal stem cells.

4. The composition of claim 2, wherein the one or more cell types comprises mesenchymal stem cells.

5. The composition of claim 1, wherein the partially ordered polypeptide comprises at least one chemically-interactive sequence.

6. The composition of claim 5, wherein the chemically-interactive sequence is arginylglycylaspartic acid (RGD) sequence.

7. The composition of claim 5, wherein the at least one chemically-interactive sequence is incorporated from the N terminus and/or the C terminus of the partially ordered polypeptide.

8. The composition of claim 5, wherein the at least one chemically-interactive sequence is incorporated between the N terminus and the C terminus of the partially ordered polypeptide.

9. The composition of claim 1, wherein the matrix forms a porous network when heated above a transition temperature.

10. The composition of claim 8, wherein a concentration of the partially ordered polypeptide within the porous network is between about 0.2 mg/ml and about 300 mg/ml.

11. The composition of claim 1, wherein the partially-ordered polypeptide comprises a plurality of structured domains, and a plurality of disordered domains.

12. The composition of claim 11, wherein the partially ordered polypeptide is photo-crosslinkable.

13. The composition of claim 11, wherein at least one of the plurality of disordered domains comprises a plurality of an amino acid sequence of (GXGVP)ₙ, wherein X is any amino acid except proline and n is an integer greater than or equal to 1, and the structured domain comprises a polyalanine domain.

14. The composition of claim 11, wherein the disordered domain comprises a plurality of an amino acid sequence of (GXGVP)ₙ, wherein X is Val, or Ala, or a mixture of Ala and Val, and wherein n is an integer from 1 to 50.

15. The composition of claim 13, wherein the polyalanine domain comprises (Ala)ₘ, wherein m is an integer from 5 to 50.

16. The composition of claim 13, wherein the polyalanine domain comprises one or more of:
(A)₂₅;
K(A)₂₅K;
D(A)₂₅K;
GD(A₂₅)K; or
GK(A₂₅)K.

17. The composition of claim 1, wherein the matrix has a transition temperature of heating (Tt-heating) and a transition temperature of cooling (Tt-cooling).

18. The composition of claim 17, wherein the transition temperature of cooling (Tt-cooling) is concentration-independent.

19. The composition of claim 17, wherein the transition temperature of heating (Tt-heating) and the transition temperature of cooling (Tt-cooling) range from about 10° C. to about 45° C.

20. The composition of claim 17, wherein the matrix forms a solid aggregate above the Tt-heating.

21. The composition of claim 20, wherein the solid aggregate resolubilizes when cooled to below the Tt-cooling.

22. The composition of claim 20, wherein the solid aggregate is a stable three-dimensional matrix.

23. The composition of claim 20, wherein the solid aggregate comprises a plurality of micropores.

24. The composition of claim 1, wherein the composition comprises between about 200 pM and about 2 mM of the matrix.

25. The composition of claim 1, wherein the composition is a shapeable liquid or semisolid.

26. The composition of claim 1, wherein the composition is injectable or implantable.

27. The composition of claim 1, wherein the composition is shapeable or moldable into 2- or 3-dimensional shapes, areas, or volumes.

28. The composition of claim 1, wherein the matrix permits cell infiltration and vascularization.

29. A method of three-dimensional cell culture and/or organoid culture, comprising:
mixing, at a first temperature, a plurality of cells with a phase-changing matrix comprising a partially ordered polypeptide;
heating the mixture to a second temperature that is higher than the first temperature, wherein the phase-changing matrix forms a porous network when heated to the second temperature, the porous network having a three-dimensional structure comprising the phase-changing matrix and the plurality of cells, the plurality of cells being encapsulated therein; and
culturing the solid aggregate at physiological conditions.

30. The method of claim 29, wherein the plurality of cells comprises one or more cell types.

31. The method of claim 30, wherein the one or more cell types comprises at least one cell type selected from the group consisting of: bone-marrow derived mesenchymal stem cells and adipose-derived mesenchymal stem cells.

32. The method of claim 30, wherein the one or more cell types comprises mesenchymal stem cells.

33. A method of extracting a plurality of cells from three-dimensional cell culture, comprising:
cooling, to a first temperature, a mixture of the plurality of cells and a phase-changing matrix comprising a partially ordered polypeptide, the phase-changing matrix solubilizing at the first temperature; and
extracting the plurality of cells from the solubilized phase-changing matrix.

34. A method for tissue replacement and/or augmentation, comprising:
mixing, at a first temperature, a plurality of cells with a phase-changing matrix comprising a partially ordered polypeptide;
heating the mixture to a second temperature that is higher than the first temperature, wherein the phase-changing matrix forms a porous network when heated to the second temperature, the porous network having a three-dimensional structure comprising the phase-changing matrix and the plurality of cells, the plurality of cells being encapsulated therein; and
implanting the solid aggregate into a subject.

35. A method for tissue replacement and/or augmentation, comprising:
mixing, at a first temperature, a plurality of cells with a phase-changing matrix comprising a partially ordered polypeptide;
heating the mixture to a second temperature that is higher than the first temperature, wherein the phase-changing matrix forms a porous network when heated to the second temperature, the porous network having a three-dimensional structure comprising the phase-changing matrix and the plurality of cells, the plurality of cells being encapsulated therein;
culturing the solid aggregate at physiological conditions; and
implanting the solid aggregate into a subject at a target region.

36. A method for tissue replacement and/or augmentation, comprising:
mixing, at a first temperature, a plurality of cells with a phase-changing matrix comprising a partially ordered polypeptide; and
injecting the mixture into a subject at a target region, the phase-changing matrix forming a porous network when heated at least to a second temperature greater than the first temperature, the second temperature being less than or equal to body temperature, wherein the porous network has a three-dimensional structure comprising the phase-changing matrix and the plurality of cells, the plurality of cells being encapsulated therein.

37. The composition of claim 1, wherein the plurality of cells encapsulated in the matrix form at least one organoid.

38. The method of claim 29, wherein the cultured plurality of cells forms an organoid.

39. A method of two-dimensional cell culture, comprising:
depositing, at a first temperature, a phase-changing matrix comprising a partially ordered polypeptide onto a culture surface;
heating the deposited phase-changing matrix to a second temperature, wherein the phase-changing matrix forms a porous network when heated to the second temperature;
seeding a plurality of cells onto a surface of the porous network; and
culturing the plurality of cells.

40. The method of claim 39, further comprising:
cooling the phase-changing matrix to the first temperature such that the phase-changing matrix solubilizes; and
extracting the cultured plurality of cells from the solubilized phase-changing matrix.
